(19) United States
(12) Patent Application Publication
(10) Pub. No.: US 2003/0166303 A1
(43) Pub. Date: Sep. 4, 2003

(54) Novel screening method for molecular antagonist using flow-cytometry

(76) Inventors: David Peritt, Bala Cynwyd, PA (US); Jonathan H. Seideman, Southampton, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/322,680

(22) Filed: Dec. 18, 2002

Related U.S. Application Data
(60) Provisional application No. 60/341,748, filed on Dec. 21, 2001.

(51) Int. Cl. G01N 33/53; G01N 33/567; G01N 33/543

(52) U.S. Cl. 436/523; 435/7.5

(57) ABSTRACT

The present invention relates to a robust and sensitive assay system utilizing synthetic spherical structures in place of cells for a FACS analysis. The method and system detects the affinity and neutralization activity of a biological molecule without interference from growth medium or cell supernatants. The systems associated with these methods allow high-throughput screening of assays for, in particular, neutralizing monoclonal antibodies.

### FIELD OF INVENTION

The present invention relates to an assay system utilizing synthetic spherical structures and flow cytometry analysis to determine the binding affinity between two biological molecules and screen potential inhibitors of biologically relevant interactions.

### BACKGROUND OF INVENTION

Development of therapeutic proteins often requires the screening of numerous potential candidates to select one with the appropriate effect as well as affinity for the targeted molecule. For example, the selection of therapeutic monoclonal antibodies often requires the screening of numerous antibody candidates in order to select an appropriate neutralizing antibody with strong enough affinity to maximize efficacy in vivo.

Current screening approaches include, for example, collecting hybridoma supernatants containing antibody and testing for ligand binding by coating polypropylene 96-well plates with an anti-F_{c} antibody, incubating supernatants with coated plates for capture, then probing plates with a labeled antigen to assess binding activity. Although these types of assays are sensitive, the cell systems used are often susceptible to at least changes in pH, passage number, endotoxin, and most notably, extraneous biomolecules such as cytokines. Because of this susceptibility, it is generally necessary for an antibody candidate to be fully purified before screening; however, splenic fusions often result in numerous antibody-producing hybridomas, compounding the effort required to purify and test each candidate. The instant invention addresses the problems in the current art, by avoiding cell-based assays while providing relevant data. More specifically, the present invention provides for the affinity and neutralization screening of potential therapeutic proteins using synthetic nicrospheres and flow-cytometry analysis (FACS).

### SUMMARY OF THE INVENTION

The objects of the present invention may minimize problems associated with cell-based FACS analysis by using synthetic microspheres coupled to proteins of interest, such as antibodies, antigens, ligands and receptors, to obtain relevant data. In particular, an object of the present invention includes assaying the natural interactions between therapeutic protein candidates and target molecules while avoiding extraneous factors associated with cell culture. An additional object may be to simultaneously measure multiple analytes through use of synthetic microspheres.

To achieve the objects and in accordance with the purpose of the invention, as embodied and broadly described herein, the present invention may include, in one or more embodiments, a method for determining the affect of a biological molecule on the binding of two proteins using flow-cytometry comprising combining a first protein-coupled cell-substitute including a first protein coupled to a cell-substitute; a second protein that is capable of being labeled with a fluorescent marker, wherein the first and second proteins bind to each other, and; titrated amounts of a biological that competes with he second protein for binding with the first protein or that inhibits the second protein from binding to the first protein, such that unbound protein is removed; then analyzing the binding of said first protein to said second protein by FACS analysis.

Another embodiment of the invention provides a method for screening protein candidates for competing activity against a known protein, using flow-cytometry by coupling a first protein to a cell-substitute, combining the protein-coupled cell-substitute with fixed amounts of a second protein that is capable of being labeled with a fluorescent marker and that is known to bind with the first protein, adding titrated amounts of a third biological molecule that may compete with or inhibit the second protein for binding with the first protein, washing away any unbound protein, and analyzing the binding of the first protein to the second protein by FACS analysis. In another embodiment of the invention, the activity of the third biological molecule is compared with that of a known molecule.

Yet another embodiment of the invention provides a method for determining the affinity between two proteins using flow-cytometry by coupling a first protein to a cell-substitute, combining the protein-coupled cell-substitute with titrated amounts of a second protein that is capable of being labeled with a fluorescent marker, washing away any unbound protein, and analyzing the binding affinity of the first protein to the second protein by FACS analysis. In this embodiment, the first and second proteins bind to each other.

In a particular embodiment, the first protein as a receptor, the second protein is a labeled ligand, and the third biological molecule is an antibody. In another embodiment of the invention, the competition assay is employed to analyze the third protein for affinity to the ligand (or receptor, if it antagonistic). In another embodiment, the activity of the third molecule is compared to that of a known antibody.

In another embodiment of the present invention, the cell-substitute is a synthetic spherical structure. In another embodiment, the cell-substitute is a microsphere. In one embodiment of the present invention, the first protein is an antibody or a functional equivalent of an antibody and the second protein is an antigen or a functional equivalent of an antigen. In another embodiment, the first protein is an antigen or the functional equivalent of an antigen and the second protein is an antibody or a functional equivalent of an antibody. In yet another embodiment, the first protein is a receptor or the functional equivalent of a receptor and the second protein is a ligand or the functional equivalent of a ligand. In another embodiment, the first protein is a ligand or the functional equivalent of a ligand and the second protein is a receptor or the functional equivalent of a receptor.

In one embodiment of the present invention, the second protein is biotinylated. In another embodiment, the fluorescent marker is phycoerythrin conjugated with streptavidin.

In another embodiment of the present invention may include a method for conducting high throughput screening of candidate therapeutic proteins or peptides by coupling a first protein to a cell-substitute, combining the protein-coupled cell-substitute with titrated amounts of a second protein that is capable of being labeled with a fluorescent marker, washing away any unbound protein, and analyzing the binding affinity of the first protein to the second protein by FACS analysis. In this embodiment, the first and second proteins bind to each other.

### BRIEF DESCRIPTION OF THE DRAWINGS

FIG. 1a depicts the binding affinity (“K_{d}”) and maximum binding (“Bₘₐₓ”) plot of biotin-hIL-6 and immobilized hIL-6sR. This reaction used 5000 hIL-6sR-coupled microspheres per well, incubated with increasing amounts of biotin-hIL-6, each in duplicate. Binding was calculated using a SA-PE probe and measuring the median fluorescence intensity in a Luminex 100 reader. The Y axis indicates median fluorescence intensity (MFI), the X axis indicates concentration. A non-linear regression curve was calculated using the GraphPad Prism one-site hyperbola model. The inset of FIG. 1a presents a Scatchard analysis of same data. Linear regression was calculated using GraphPad Prism.

FIG. 1b depicts the inhibition of biotin-hIL-6 binding to immobilized hIL-6sR by an anti-hIL-6 monoclonal antibody. Human IL-6sR-coupled microspheres were incubated along with constant biotin-hIL-6 (20 ng/mL) and increasing amounts of anti-hIL-6 antibody (square) or isotype control (triangle), each in duplicate. Non-linear regression was calculated using the GraphPad Prism the sigmoidal dose-response model with variable slope.

FIG. 1c depicts the inhibition of biotin-hIL-6 binding to immobilized hIL-6sR by an anti-hIL-6 monoclonal antibody in various media. Human IL-6sR-coupled microspheres were incubated along with biotin-hIL-6 (5 ng/mL) and increasing amounts of anti-hIL-6 antibody in Luminex assay buffer (square, dotted line), IMDM with Origen (triangle, dashed line), or isotype control in IMDM (inverted triangle, solid line), each in duplicate. Non-linear regression was calculated using the GraphPad Prism sigmoidal dose-response model with variable slope.

FIG. 2a depicts the results of a 7TD1 assay, showing relative activities of IL-6 species. Increasing amounts of recombinant murine IL-6 (inverted triangle, dotted line), recombinant human IL-6 (square, hatched line), or biotin-human IL-6 (triangle, solid line) were incubated along with 200 7TD1 cells per well for 72 hours at 37° C. Proliferation was assayed by ATPLite (Packard). The Y axis indicates photons per second, the X axis indicates the concentration of IL-6. Non-linear regression was calculated using the GraphPad Prism sigmoidal dose-response model with variable slope.

FIG. 2b depicts the inhibition of IL-6-dependent cell proliferation by an anti-hIL-6 monoclonal antibody in prepared media. Constant recombinant human IL-6 (250 pg/mL) was incubated along with 200 7TD1 cells as above with increasing amounts of anti-hIL-6 antibody in either IMDM (square, hatched line) or IMDM with Origen (circle, solid line), or isotype control in IMDM (inverted triangle, dotted line), each in duplicate. Proliferation was assayed by ATPLite. Non-linear regression was calculated using the GraphPad Prism sigmoidal dose-response model with variable slope.

### DETAILED DESCRIPTION OF THE INVENTION

It is to be understood that this invention is not limited to the particular methodology, protocols, constructs, formulae and reagents described and as such may vary. It is also to be understood that the terminology used herein is for the purpose of describing particular embodiments only, and is not intended to limit the scope of the present.

It must be noted that as used herein and in the appended claims, the singular forms “a,” “and,” and “the” include plural reference unless the context clearly dictates otherwise. Thus, for example, reference to “an antibody” is a reference to one or more antibodies and includes equivalents and functional portions thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventor is not entitled to antedate such disclosure by virtue of prior invention.

Proteins, as the term is used herein, include any protein, polypeptide, peptide, amino acid sequence, or fragment or analog thereof. Hence, the term protein, as used herein and in the appended claims, is used for convenience and in a non-limiting fashion. Such proteins include those with therapeutic or diagnostic potential, such as, without limitation, an immunoglobulin or a fragment thereof, a cytokine, a chemokine, an integrin, an antigen, a growth factor, a cell cycle protein, a hormone, a neurotransmitter, a receptor or fusion protein thereof, a blood protein, an antimicrobial, any fragment thereof, an antagonist or agonist of any of the foregoing, or any structural or functional analog of any of the foregoing.

Biological molecules useful in the present invention include, for example, a protein, a fatty acid or lipid, a carbohydrate or oligosaccharide, a nucleic acid, a chemical, or any other biologically relevant molecule. Examples of these biological molecules include without limitation, an immunoglobulin or a fragment thereof, a cytokine, a chemokine, an integrin, an antigen, a growth factor, a cell cycle protein, a hormone, a neurotransmitter, a receptor or fusion protein thereof, a blood protein, an antimicrobial, any fragment thereof, an antagonist or agonist of any of the foregoing, or any structural or functional analog of any of the foregoing. In a particular embodiment of the invention, the biological molecule is an antagonist. In another embodiment of the invention, the biological molecule is a monoclonal antibody or a functional equivalent thereof.

The present invention addresses the problems in the known approaches to determining the affinity and other activities of culture-derived therapeutic protein candidates that use whole-cell assay systems. More specifically, interfering factors associated with cell culture are avoided by replacing cell-based assays with cell-substitutes, e.g., synthetic microspheres coupled to proteins of interest. FACS is particularly preferred to obtain relevant data, although other assay systems may benefit from the approach described herein. The systems and methods of the present invention are designed to assay the natural interactions between therapeutic protein candidates and target biomolecules without intervening extraneous factors associated with cell culture.

The use of synthetic microspheres in FACS also allows the simultaneous measurement of multiple analytes. Additionally, these microspheres provide fluorescent articles comprising a core or carrier particle having on its surface a plurality of smaller polymeric particles or nanoparticles, which may be stained with different fluorescent dyes. When excited by a light source, the particles are capable of giving off multiple fluorescent emissions simultaneously, which is useful for multiplexed analysis of a plurality of analytes in a sample. See, e.g., U.S. Pat. No. 6,268,222.

Development of a therapeutic protein, such as a monoclonal antibody, receptor or other ligand, often requires the screening of numerous candidates in order to select an appropriate activity. For example, under the current approach regarding the neutralizing activity of a monoclonal antibody candidate, hybridoma supernatants containing antibody are tested for ligand binding by coating polypropylene 96-well plates with an anti-F_{c} antibody, incubating supernatants with coated plates for capture, and then probing plates with a labeled antigen to assess binding activity. Although this is a reliable method for screening, the assay only selects antibodies that bind, but do not necessarily neutralize, the target antigen. Assessing biological neutralization activity is a critical second step which normally requires a more complex screening process.

Typically, neutralization or other desired activity is demonstrated in a biologically relevant system, such as a cell-based assay. Inhibition of assay endpoint, whether it be cytokine secretion, cell migration, or proliferation, can then be assayed as a measure of candidate, e.g., antibody, efficacy. While these types of assays are very sensitive, cell systems are often susceptible to, among other things, changes in pH, passage number, endotoxin, and most notably, extraneous biomolecules such as cytokines. Schwabe et al., 168(1) CELL IMMUNOL. 117-21 (1996). Because of this susceptibility, it is often necessary that a candidate biological molecule, such as an antibody candidate be fully purified before screening. However, splenic fusions often result in numerous antibody-producing hybridomas, compounding the effort required to purify and test each candidate. This can be a time-consuming and cumbersome process.

The present invention provides an assay that circumvents the inherent problems of current cell-based assay approaches, yet remains biologically relevant. More specifically, the present invention provides for technologies, using FACS of a protein-coupled cell-substitute, that measure the natural interaction between receptor and ligand, and are not affected by extraneous factors.

The present invention uses traditional flow-cytometry hardware and spectrally discrete polystyrene beads, or microspheres, to measure multiple analytes simultaneously. Flow cytometry is an optical technique that analyzes particular particles in a fluid mixture based on the particles' optical characteristics by using an instrument known as a flow cytometer. Background information on flow cytometry may be found in SHAPIRO, PRACTICAL FLOW CYTOMETRY (3rd ed., Alan R. Liss, Inc., 1995) (hereinafter SHAPIRO), and MELAMED ET AL., FLOW CYTOMETRY & SORTING, (2nd ed., Wiley-Liss, 1990) (hereinafter MELAMED). Flow cytometers hydrodynamically focus a fluid suspension of particles into a thin stream so that the particles flow down the stream in substantially single file and pass through an examination zone. A focused light beam, such as a laser beam, illuminates the particles as they flow through the examination zone. Optical detectors within the flow cytometer measure certain characteristics of the light as it interacts with the particles. Commonly used flow cytometers such as the Becton-Dickinson Immunocytometry Systems “FACSCAN” can measure forward light scatter (generally correlated with the refractive index and size of the particle being illuminated), side light scatter (generally correlated with the particle's size), and particle fluorescence at one or more wavelengths. Fluorescence is typically imparted by incorporating, or attaching a fluorochrome within the particle. Flow cytometers and various techniques for their use are described in, generally, SHAPIRO, MELAMED, and U.S. Pat. Nos. 5,981,180; 6,046,807; and 6,139,800.

Another example of commercially available hardware and microspheres is the Luminex-100 flow system, by Luminex Corp., (Austin, Tex.). See U.S. Pat. Nos. 5,981,180; 6,046,807; and 6,139,800. Previously, this system had been used in both sandwich ELISA and DNA hybridization formats. For instance, one antibody, immobilized on a bead of a particular spectral address, captures analyte. A secondary biotin-labeled antibody, in combination with streptavidin-phycoerythrin (SA-PE), serves as a reporter. Oliver et al., 44(9) CLIN. CHEM. 2057-60 (1998). Any labeling dye may be used however, such as those described in U.S. Pat. No. 6,268,222. Analogous to the ELISA format, DNA hybridization requires immobilization of DNA probe sequences and use of biotin-labeled PCR products to detect a specific mutation. Dunbar & Jacobson, 46(9) CLIN. CHEM. 1498-500 (2000).

In this format, FACS of protein-coupled microspheres offers sensitivity comparable to traditional ELISA and DNA hybridization techniques known in the art, such as in U.S. Pat. Nos. 5,736,330 and 6,057,107, as well as significantly shorter assay times. Vignali, 243(1-2) J. IMMUNOL. METS. 243-55 (2000). This is an obvious advantage over cell-based systems, which require up to three days to obtain a measurable output. Because of the demonstrated efficiency and sensitivity in the ELISA and hybridization formats, in addition to the time-saving aspects of this system, this system allows for direct assessment of the interaction between receptor and ligand.

The microspheres of the present invention may be made of polystyrene, or any polymer that can be made into a roughly spherical shape or bead of the size required for FACS readings. Such microspheres, preferably, should be suitable for coupling to a protein useful in the FACS assay. Such coupling may be achieved by any suitable chemistry. Such microspheres, proteins, and coupling chemistries are know in the art (see e.g., U.S. Pat. No. 6,268,222) and the invention herein described is not limited to any particular known or yet-to-be-discovered microspheres, proteins, and coupling chemistries. Similarly, any suitable FACS equipment may be used in the present invention, and several varieties of instruments and associated software are known and available to those of ordinary skill in the art. Indeed, the methods described herein may be adapted to many receptor-ligand reactions, as long as the one of the two is available in a soluble form, one of the two can be conjugated to biotin or a suitable marker without loss of biological activity, and the two species are sufficiently stable to withstand assay conditions.

Another embodiment of the invention provides a method for screening protein candidates for competing activity against a known protein, using FACS, by coupling a first protein to a cell-substitute, combining the protein-coupled cell-substitute with fixed amounts of a second protein that is capable of being labeled with a fluorescent marker and that is known to bind with the first protein, adding titrated amounts of a third biological molecule that may compete with or inhibit the second protein for binding with the first protein, such as an antagonist, and analyzing the binding of the first protein to the second protein by FACS analysis.

In one embodiment of the invention, the first protein as a receptor, the second protein is a labeled ligand, and the third biological molecule is an antibody. In another embodiment of the invention, the competition assay is employed to analyze the third biological molecule for affinity to the ligand (or receptor, if it antagonistic). The biological molecule of this assay may be a protein, a fatty acid or lipid, a carbohydrate or oligosaccharide, a nucleic acid, a chemical, or any other biologically relevant molecule.

In a particular embodiment of the invention, a receptor molecule may be coupled to a microsphere, incubated with a biotin-labeled ligand, and titrated against an anti-ligand antibody that inhibits the interaction between the receptor and the ligand. Binding may be assessed using an appropriate reporter. This assay is particularly useful for detection and characterization of a neutralizing monoclonal antibody. In a particular example of this embodiment of the invention, discussed in further detail below, the human interleukin-6 soluble receptor (hIL-6sR) was coupled to Luminex polystyrene microspheres, incubated along with a biotin-labeled recombinant human interleukin-6 (rhIL-6) ligand, titrated against anti-hIL-6 antibody, and the binding interactions assessed using a streptavidin-phycoerythrin reporter.

The present invention offers sensitivity comparable to traditional Enzyme-Linked Immunosorbent (“ELISA”) assays in significantly shorter assay times. This is an obvious advantage over cell-based systems, which may require up to three days to obtain a measurable output. Assay time is reduced by using a high-throughput screening (HTS) application. In one embodiment, the entire assay may consist of one 45 minute incubation period with neutralizing antibody and a biotinylated ligand, followed by a 20 minute incubation with a phycoerythin reporter. The entire assay may be completed in less than two hours. Thus, in a specific embodiment of the invention, the methodologies taught herein are applied to HTS for candidate proteins or peptides.

The present invention thus allows the screening of complex, unpurified antibody samples, which are not commonly amenable to assaying, and also eliminates the purification process that usually precedes the neutralization determination step. Generally, purifying an antibody from culture is a time-consuming process because it is necessary to grow large volumes of supernatant for a small amount of antibody. The present invention circumvents this process, reducing the risk of lost hybridoma clones, and saving valuable resources by demonstrating antibody efficacy before purification.

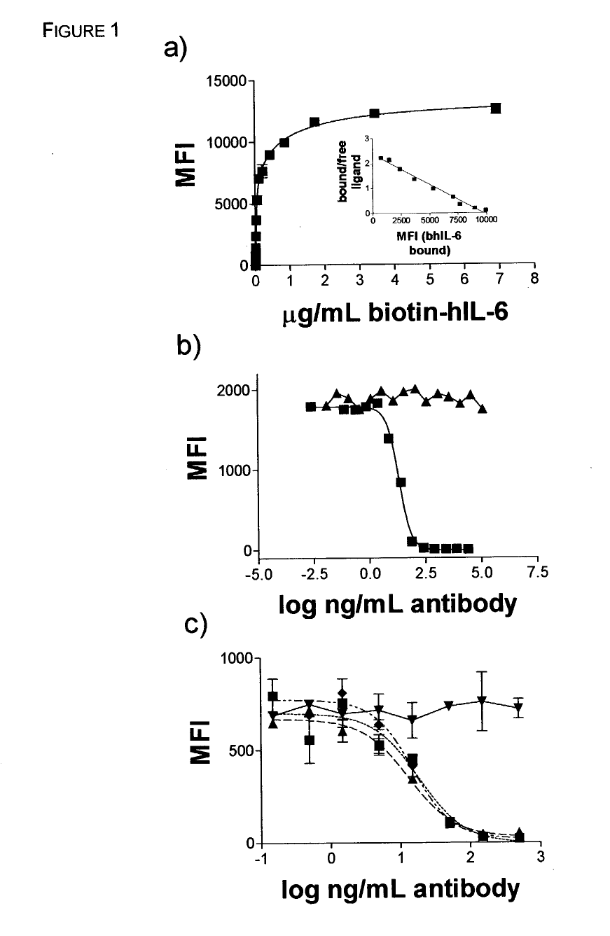

In one embodiment of the invention, a binding reaction consisting of hIL-6sR-coupled Luminex microsphere, titrated biotin-hIL-6, and a probe of streptavidin-phycoerythrin yielded appreciable fluorescent signal at biotin-IL-6 concentrations less than 0.5 ng/mL. The K_{d} for this reaction was 3.6×10⁻⁹ M (75 ng/mL). As illustrated by the nonlinear regression-derived Bₘₐₓ hyperbola, this binding was specific and saturable (FIG. 1a). Specific activity was not known for phycoerythrin fluorescence, nor for biotin-IL-6, therefore Bₘₐₓ could not be calculated. Consequently, it was not possible to determine the number of hIL-6sR molecules coupled to each microsphere.

In another embodiment of the invention biotin-IL-6 was reacted at a constant concentration for assessment of antibody competition. Twenty-one ng/mL (IC₅₀) of anti-human IL-6 monoclonal antibody displaced 50% of the maximum bound biotin-hIL-6 at 20 ng/mL (FIG. 1b). This competition method resulted in a crisply delineated sigmoidal curve with little error associated among data points (FIG. 1b). The upper asymptote of the anti-hIL-6 inhibition curve had a coefficient of variation (CV) of 7.8%, and the lower asymptote, 16%.

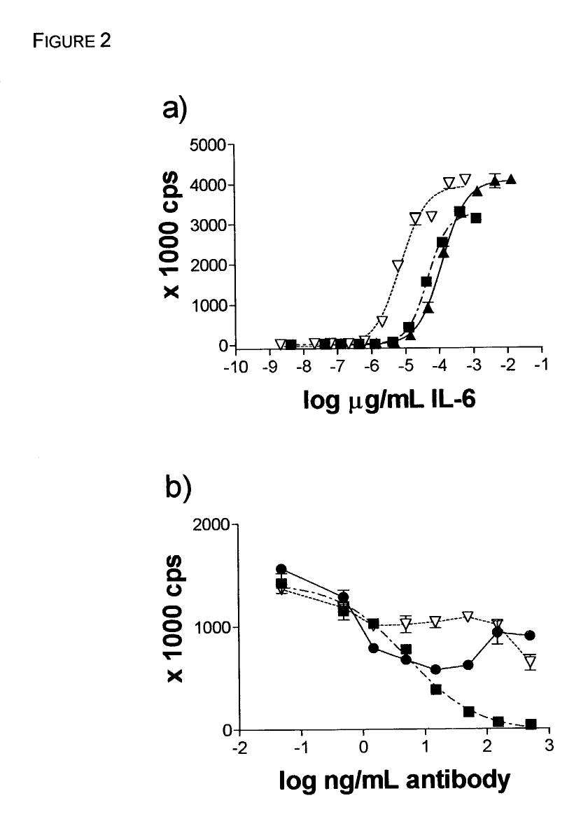

In comparison to whole-cell-based assays, at 20 ng/mL biotin-IL-6, the microsphere-based assay of the present invention showed a markedly higher signal to noise ratio than that of the 7TD1 assay (37:1 for 7TD1 bioassay, 250 pg/mL hIL-6, 176:1 for microsphere assay, 20 ng/mL IL-6, FIG. 1b and FIG. 2b). This value is defined as the ratio between the mean y-axis value of the upper asymptote of the sigmoid and the mean y-axis value of the lower asymptote. This resultant high ratio, along with the near absence of derogation along the asymptotic portions of the inhibition curve, indicates that the amount of biotin-IL-6 required to detect antibody neutralization may be decreased, increasing assay sensitivity.

In order to demonstrate this potential for increased sensitivity and simultaneously prove the robust nature of the assay, anti-human IL-6 neutralizing monoclonal antibody was prepared in either medium containing 3% Origen (a commonly used hybridoma growth supplement) or in the 1% BSA/PBS assay buffer, then titrated along with 5 ng/mL biotin-hIL-6. The two curves representing antibody in the described medium conditions are virtually indistinguishable, illustrating the microsphere assay method of the present invention may be unsusceptible to extraneous factors (FIG. 1c). At 5 ng/mL, biotin-IL-6 still binds with a signal to noise ratio of 16:1, which is sufficient to overcome any associated noise and to detect neutralizing antibody at even lower levels. The IC₅₀ of the anti-hIL-6 in this case is 11 ng/mL, while antibody can be easily detected at concentrations below 10 ng/mL. Sensitivity was increased nearly two-fold by lowering the biotin-IL-6 concentration.

In sharp contrast to the robust nature of the microsphere assay of the present invention, the 7TD1 assay, an IL-6-dependent cell proliferation assay, was affected by analogous medium conditions. As described in the examples below, an anti-human IL-6 monoclonal antibody was prepared with medium containing 3% Origen, or in normal 7TD1 growth medium (which does not contain growth supplement) and then titrated along with 250 pg/mL rhIL-6. The assay worked well in the Origen-free case, but at high concentrations, neutralization activity of anti-human IL-6 antibody in Origen containing medium was completely obscured (FIG. 2b). This sort of spurious result may be eliminated by using the microsphere receptor-binding method of the present invention.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

Microsphere Coupling

Carrier-free human interleukin-6 soluble receptor (hIL-6sR), obtained from R&D Systems (Minneapolis, Minn.), was dissolved in 500 μL coupling buffer (Dulbecco's PBS without Calcium or Magnesium, from J R H Biosci., Lenexa, Kans.) to a final concentration of 250 mg/mL. A stock vial of Luminex microspheres (Luminex Corp., Austin, Tex.) was centrifuged at 14,000×g for one minute at room temperature. In order to minimize aggregation of microspheres, pellet was sonicated until visibly disrupted then gently vortexed for 10 seconds. A 200 μL (2.5×10₆ microspheres) bolus was transferred from the stock vial to a 1.5 mL polypropylene microcentrifuge tube (Fisher Scientific, Pittsburgh, Pa.) and centrifuged as before. After gently removing supernatant, microspheres were washed twice in 80 μL of activation buffer (0.1 M Sodium Phosphate, pH 6.2). The pellet was then resuspended in 80 μL of activation buffer. N-hydroxysulfosuccinimide sodium salt (Sulfo-NHS) and 1-Ethyl-3-(3-dimethylaminopropyl) carbodiimide hydrochloride (EDC) (Pierce Chemical, Rockford, Ill.), both at 50 mg/mL in activation buffer, were prepared immediately before adding 10 μL of each to the 80 μL microsphere suspension. Microspheres were incubated at room temperature for 20 minutes in the dark. After incubation, activated microspheres were centrifuged and the supernatant removed.

The pellet was washed twice with 500 μL coupling buffer and 500 μL of hIL-6sR solution was added. In order to completely homogenize the mixture, pellet was sonicated and vortexed briefly. This mixture was rotated for two hours at room temperature in the dark. After incubation, the mixture was centrifuged and supernatant was removed. The pellet was then washed twice with milliliter aliquots of wash buffer (Dulbecco's PBS without Magnesium or Calcium, 0.05% w/v Tween 20). Finally, the pellet was resuspended in 200 μL blocking/storage buffer (DPBS without calcium or magnesium, 10 mg/mL BSA, and 0.05% w/v sodium azide). The microspheres were enumerated by hemacytometer. Approximately seventy percent recovery was typical efficiency for microsphere conjugation. Microspheres were stored in blocking buffer at 4° C. until ready for use.

### Example 2

Biotinylation of Human IL-6

Lyophilized recombinant human IL-6 (hIL6) (R&D Systems, Minneapolis, Minn.) was reconstituted with ddH₂O for a final concentration of 1.05 mg/mL. The hIL-6 solution was dialyzed (SLIDE-A-LYZER™ from Pierce Chemical, Rockford, Ill.) (10,000 MWCO) against bicarbonate buffer (200 mM NaHCO₃, 150 mM KCl, pH 8.4) overnight at 4° C. After buffer exchange, 105 μL of the hIL-6 solution was transferred to a microcentrifuge tube containing an equivalent of bicarbonate buffer. Sulfo-NHS-biotin (Pierce Chemical) was prepared in double-distilled water (ddH₂O) at a concentration of 9.0×10⁻³ M (4 mg/mL), immediately before adding 4 μL (3.7×10⁹ mol) to a tube containing 210 μL of the hIL-6 solution for final concentrations of 1.7×10⁻⁴ M and 2.4×10⁻⁵ M, for sulfo-NHS-biotin and hIL-6, respectively. The solution was then vortexed and incubated at room temperature in the dark with gentle shaking for 35 minutes. After incubation, 21 μL of 1 M NH₄Cl solution was added to quench the reaction. Quenched reaction was dialyzed against Dulbecco's PBS at 4° C. for eight hours and then immediately combined with an equivalent of 0.2% BSA/DPBS to prevent adsorption of ligand to vessel walls. This volume was then transferred to a new SLIDE-A-LYZER™ cartridge for further dialysis. Dialysis was continued at 4° C. overnight. Biotinylated hIL-6 was stored at −70° C. until ready for use. Biotin-hIL-6 was shown to retain its biological activity by the 7TD1 bioassay, depicted in FIG. 2a.

### Example 3

Estimation of Binding Constant (K_{d}) of Biotin-IL-6 for IL-6 Receptor-Microsphere

All reactions were completed in a 96-well filter plate (Millipore, Bedford, Mass.) that allowed for washing of the Luminex microspheres by placing on a vacuum manifold. A filter plate was washed twice with 100 μL blocking/storage buffer. In duplicate, increasing amounts of biotin-IL-6 in 50 μL blocking/storage buffer were added to each well. After sonication, 50 μL of IL-6sR coupled microspheres in blocking/storage buffer (˜5000 microspheres/well) were added to all wells. The reaction was incubated in the dark for forty-five minutes at room temperature with gentle shaking. The filter plate was then washed as before. Finally, 100 μL of 0.5 mg/mL streptavidin-phycoerythrin (SA-PE) was added to all wells (BD Pharmingen, San Diego, Calif.). The filter plate was incubated as above for an additional 20 minutes. The wash was repeated by applying suction to the filter plate. The filter plate was washed a final time with DPBS immediately before 100 μL of 0.1% formaldehyde in DPBS was added to all wells. This was done in order to fix molecular interactions before reading on the Luminex-100. Fluorescence intensity was read on the Luminex-100 instrument (Luminex Corp., Austin, Tex.), counting region 054, 100 events per bead, gated from 8300-13,500 (undefined units) on the doublet discriminator.

### Example 4

Neutralization of Biotin-IL-6 by CLB8

For this competition assay, the murine anti-human IL-6 monoclonal antibody, CLB8 (as disclosed in PCT WO 91/08774), is prepared at 1 μg/mL in either blocking/storage buffer or fresh medium (RPMI 1640, 10%FBS, 2 mM L-Gln, 3% Origen). The prepared antibody is then titrated in blocking/storage buffer and combined in a 100 mL reaction containing 5000 IL-6R-coupled microspheres and either 20 ng/mL or 5 ng/mL biotin-IL-6. Plates are incubated for 45 minutes at room temperature with a gentle shake and then washed as above. SA-PE is added as described above; the development process is continued as in Example 3.

### Example 5

7TD1 Proliferation Assay

The murine B-cell myeloma, 7TD1, is cultured in IMDM, 10% FBS, 2 mM L-Glutamine at 37° C., 5% CO₂. A 100 μL reaction containing 200 7TD1 cells, a hIL-6 concentration of 250 pg/mL, and increasing amounts of CLB8 in either 7TD1 growth medium or medium containing 3% Origen, as in the assay of Example 3, is incubated for 72 hours at 37° C., 5% CO₂. In a similar fashion, increasing amounts of biotin-hIL-6, rh-IL-6, and recombinant murine interleukin-6 (rmIL-6) are added to compare bioactivities of the three species. Cell proliferation is assessed by ATPLite (Packard Instrument Co., Meriden, Conn.). This step is completed by adding 50 mL of ATPLite lysis buffer, shaking for three minutes, and subsequently adding 50 mL of ATPLite substrate and shaking for an additional minute. Chemiluminescence is measured using Topcount plate reader (Packard Instrument Co.).

We claim:

1. A method for determining the affect of a biological molecule on the binding of two proteins using flow-cytometry comprising:
combining (i) a first protein-coupled cell-substitute comprising a first protein coupled to a cell-substitute; (ii) a second protein that is capable of being labeled with a fluorescent marker, wherein said first and second proteins bind to each other; and (iii) titrated amounts of a biological molecule that competes with the second protein for binding with the first protein, or that inhibits the second protein from binding with the first protein, such that unbound protein is removed; and
analyzing the binding of said first protein to said second protein by FACS analysis.

2. The method of claim 1, wherein said cell-substitute comprises a synthetic spherical structure.

3. The method of claim 1, wherein said cell-substitute comprises a microsphere.

4. The method of claim 1, wherein said first protein is an antibody or a functional equivalent thereof.

5. The method of claim 4, wherein said second protein is an antigen or a functional equivalent thereof.

6. The method of claim 1, wherein said first protein is an antigen or a functional equivalent thereof.

7. The method of claim 6, wherein said second protein is an antibody or a functional equivalent thereof.

8. The method of claim 1, wherein said first protein is a receptor or a functional equivalent thereof.

9. The method of claim 8, wherein said second protein is a ligand or a functional equivalent thereof.

10. The method of claim 1, wherein said first protein is a ligand or a functional equivalent thereof.

11. The method of claim 10, wherein said second protein is a receptor or a functional equivalent thereof.

12. The method of claim 1, wherein said third biological molecule is a protein.

13. The method of claim 12, wherein said protein is an antibody or a functional equivalent thereof.

14. The method of claim 1, wherein said second protein is biotinylated.

15. The method of claim 1, wherein said third biological molecule is an immunoglobulin or a functional equivalent thereof.

16. The method of claim 1, wherein said fluorescent marker further comprises phycoerythrin conjugated with streptavidin.

17. A method for conducting high throughput screening of candidate biological molecules using the method of claim 1.
